(19) United States
(12) Patent Application Publication
(10) Pub. No.: US 2018/0098818 A1
(43) Pub. Date: Apr. 12, 2018

(54) HEALTH MONITORING KIT

(71) Applicant: Under Armour, Inc., Baltimore, MD (US)

(72) Inventors: Breyna Fries, Baltimore, MD (US); Brian Boring, Baltimore, MD (US); Eric Boam, Baltimore, MD (US); Evan Torchin, Baltimore, MD (US); Helene Christman, Baltimore, MD (US); Paul Pugh, Baltimore, MD (US)

(21) Appl. No.: 15/728,800

(22) Filed: Oct. 10, 2017

Related U.S. Application Data
(60) Provisional application No. 62/407,233, filed on Oct. 12, 2016.

(51) Int. Cl. A61B 50/10; B65D 1/36; B65D 43/02; B65D 51/24; A61B 50/30; A61B 5/024

(52) U.S. Cl. CPC A61B 50/10; B65D 1/36; B65D 43/02; A61B 2050/0079; A61B 50/30; A61B 5/02438; B65D 2543/00537; B65D 51/245

(57) ABSTRACT

A health monitoring kit includes a package unit, a scale, a display band, and a heart monitor. The package unit includes a base panel with a base face, a first upper panel, and a second upper panel. The first upper panel is connected to the base panel with a first hinge member, and the second upper panel is connected to the base panel with a second hinge member. The scale is retained in the base panel and covers a portion of the base face. The display band is retained in the first upper panel. The heart monitor is retained in the second upper panel.

### CROSS-REFERENCE TO RELATED APPLICATIONS

This application claims priority to U.S. Provisional Patent Application No. 62/407,233 entitled “Health Monitoring Kit,” filed Oct. 12, 2016, the entire contents of which are incorporated by reference herein.

### COPYRIGHT

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### FIELD

The system disclosed in this document relates to health and fitness monitoring devices and, more particularly, to kits used to sell and display such health and fitness monitoring devices.

### BACKGROUND

Health and fitness monitoring devices are increasingly utilized by individuals interested in tracking metrics and other health data related to their personal health and fitness. Health and fitness monitoring devices may include any of various devices such as such as heart rate monitors, step counters, stair counters, global positioning system (“GPS”) tracking devices, scales, sleep monitors, smartphones, smartwatches or wristbands, as well as various other motion tracking and/or biometric monitoring devices. Sensors in such health and fitness monitoring devices (which may also be referred to herein as “health monitoring devices” or “health tracking devices”) allow the user to easily track and automatically log health and fitness information with the health tracking device.

Health tracking devices are increasingly interconnected with users carrying multiple health tracking devices that are capable of wirelessly communicating with each other. For example, a single user may utilize a wristband or watch with a step counter during the day, add a heart rate monitor during workouts, and make use of a scale to track his or her weight on a daily basis. Each of these health tracking devices may communicate and share health data with one another or with a smartphone or other computer.

Users interested in taking advantage of multiple health tracking devices must typically purchase each health tracking device separately. This process of searching for various individual health tracking devices may be time consuming. When making this purchase decision, the user must also consider whether the various health tracking devices are capable of communicating within the same health tracking system. Furthermore, once the user purchases multiple health tracking devices, he or she must take care to store the health tracking devices in a known location, so the devices are readily available for the user when desired.

In view of the foregoing, it would be advantageous to provide a kit of health monitoring devices that are all capable of use within a single health tracking system. It would be advantageous if the kit were configured for easy and convenient in-store display of the various health tracking devices within the kit. It would also be advantageous if such a kit could be retained by the user and used to easily and conveniently store the health tracking devices following use. Moreover, it would be advantageous if the kit provided a unique experience for the user when opening and exploring the contents of the kit.

### SUMMARY

In accordance with one exemplary embodiment of the disclosure, there is provided a health monitoring kit comprising a package unit, a scale, a display band, and a heart monitor. The package unit includes a base panel with a base face, a first upper panel, and a second upper panel. The first upper panel is connected to the base panel with a first hinge member, and the second upper panel is connected to the base panel with a second hinge member. The scale is retained in the base panel and covers a portion of the base face. The display band is retained in the first upper panel. The heart monitor is retained in the second upper panel.

Pursuant to another exemplary embodiment of the disclosure, there is provided a health monitoring kit comprising a two door cabinet unit including a base, a first door, and a second door. The base defines a base volume, the first door defines a first door volume, and the second door defines a second door volume. A first door insert is provided in the first door, the first door insert including a first cavity that fills a minority of the first door volume. A second door insert is provided in the second door, the second door insert including a second cavity that fills a minority of the second door volume. A base insert is provided in the base, the base insert including a third cavity that fills a majority of the base volume. A first health tracking device is positioned in and fills a majority of the first cavity. A second health tracking device is positioned in and fills a majority of the second cavity. A third health tracking device is positioned in and fills a majority of the heart monitor cavity.

In accordance with yet another exemplary embodiment of the disclosure, there is provided a health monitoring kit comprising a package unit, a first health tracking device, a second health tracking device, and a third health tracking device. The package unit includes a base panel, a first upper panel, and a second upper panel. The first upper panel is connected to the base panel with a first hinge member, and the second upper panel is connected to the base panel with a second hinge member. The first health tracking device is retained in the base panel. The second health tracking device is retained in the first upper panel. The third health tracking device is retained in the second upper panel.

The above described features and advantages, as well as others, will become more readily apparent to those of ordinary skill in the art by reference to the following detailed description and accompanying drawings. While it would be desirable to provide a health monitoring kit that provides one or more of these or other advantageous features, the teachings disclosed herein extend to those embodiments which fall within the scope of the appended claims, regardless of whether they accomplish one or more of the above-mentioned advantageous features.

### BRIEF DESCRIPTION OF THE DRAWINGS

The foregoing aspects and other features of a health monitoring kit are explained in the following description, taken in connection with the accompanying drawings wherein:

FIG. 1 shows a top perspective view of a health monitoring kit including a package unit and a lid;

FIG. 2 shows the health monitoring kit of FIG. 1 with the lid removed from the package unit;

FIG. 3 shows a proximal side view of the package unit of FIG. 2;

FIG. 4 shows a right side view of the package unit of FIG. 2;

FIG. 5 shows a proximal perspective view of the package unit of FIG. 2 in a closed position with a left upper panel and a right upper panel in a closed position over a base panel;

FIG. 6 shows a proximal perspective view of the package unit of FIG. 5 with the left upper panel and the right upper panel moving toward an open position;

FIG. 7 shows a proximal perspective view of the package unit of FIG. 5 with the left upper panel and the right upper panel an open position, exposing health tracking devices within the package unit;

FIG. 8 shows a proximal perspective view of the package unit of FIG. 7 with the health tracking devices removed from the package unit; and

FIG. 9 shows a top view of the package unit of FIG. 8.

### DETAILED DESCRIPTION

For the purposes of promoting an understanding of the principles of the disclosure, reference will now be made to the embodiments illustrated in the drawings and described in the following written specification. It is understood that no limitation to the scope of the disclosure is thereby intended. It is further understood that the present disclosure includes any alterations and modifications to the illustrated embodiments and includes further applications of the principles of the disclosure as would normally occur to one skilled in the art which this disclosure pertains.

In the following detailed description, reference is made to the accompanying drawings which form a part hereof wherein like numerals designate like parts throughout, and in which is shown, by way of illustration, embodiments that may be practiced. It is to be understood that other embodiments may be utilized and that structural changes may be made without departing from the scope of the present disclosure. Therefore, the following detailed description is not to be taken in a limiting sense, and the scope of embodiments is defined by the appended claims and their equivalents.

Aspects of the disclosure are disclosed in the accompanying description. Alternate embodiments of the present disclosure and their equivalents may be devised without parting from the spirit or scope of the present disclosure. It should be noted that any discussion herein regarding “one embodiment”, “an embodiment”, “an exemplary embodiment”, and the like indicate that the embodiment described may include a particular feature, structure, or characteristic, and that such particular feature, structure, or characteristic may not necessarily be included in every embodiment. In addition, references to the foregoing do not necessarily comprise a reference to the same embodiment. Finally, irrespective of whether it is explicitly described, one of ordinary skill in the art would readily appreciate that each of the particular features, structures, or characteristics of the given embodiments may be utilized in connection or combination with those of any other embodiment discussed herein.

Various operations may be described as multiple discrete actions or operations in turn, in a manner that is most helpful in understanding the claimed subject matter. However, the order of description should not be construed as to imply that these operations are necessarily order dependent. In particular, these operations may not be performed in the order of presentation. Operations described may be performed in a different order than the described embodiment. Various additional operations may be performed and/or described operations may be omitted in additional embodiments.

For the purposes of the present disclosure, the phrase “A and/or B” means (A), (B), or (A and B). For the purposes of the present disclosure, the phrase “A, B, and/or C” means (A), (B), (C), (A and B), (A and C), (B and C), or (A, B and C).

The terms “comprising,” “including,” “having,” and the like, as used with respect to embodiments of the present disclosure, are synonymous.

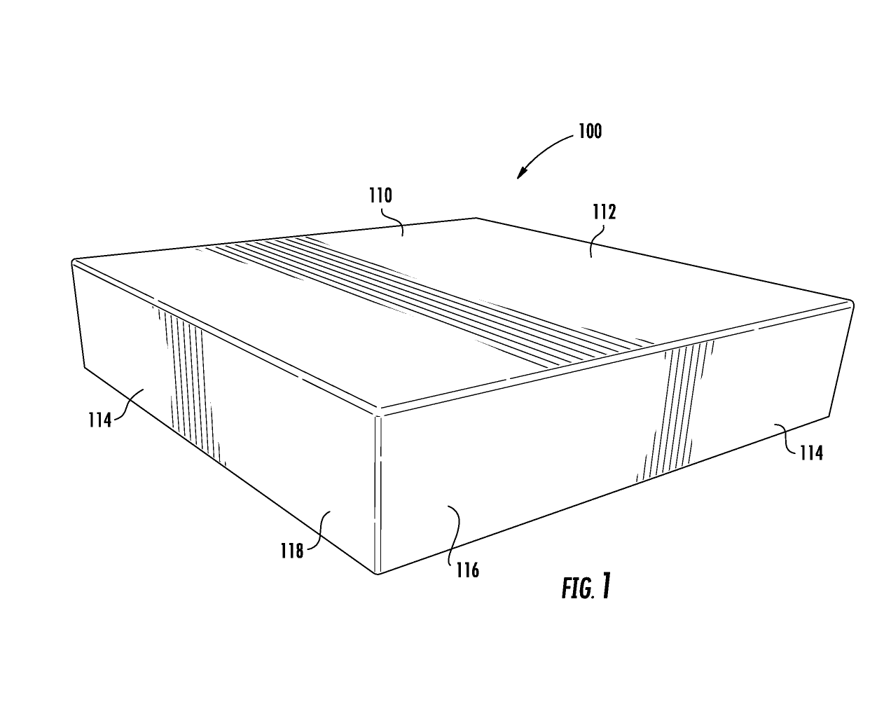

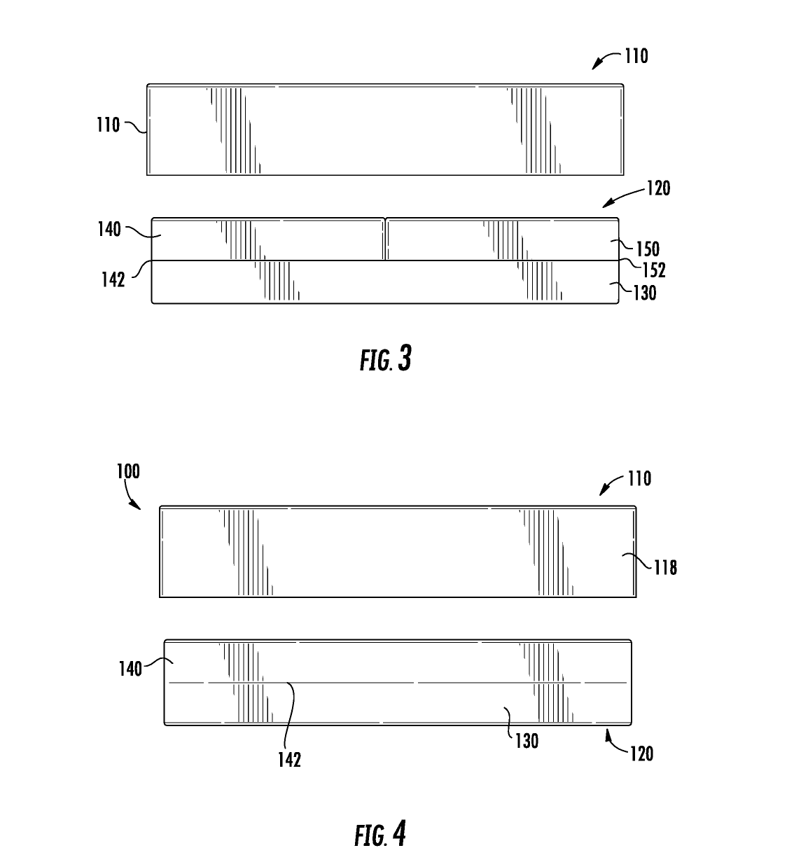

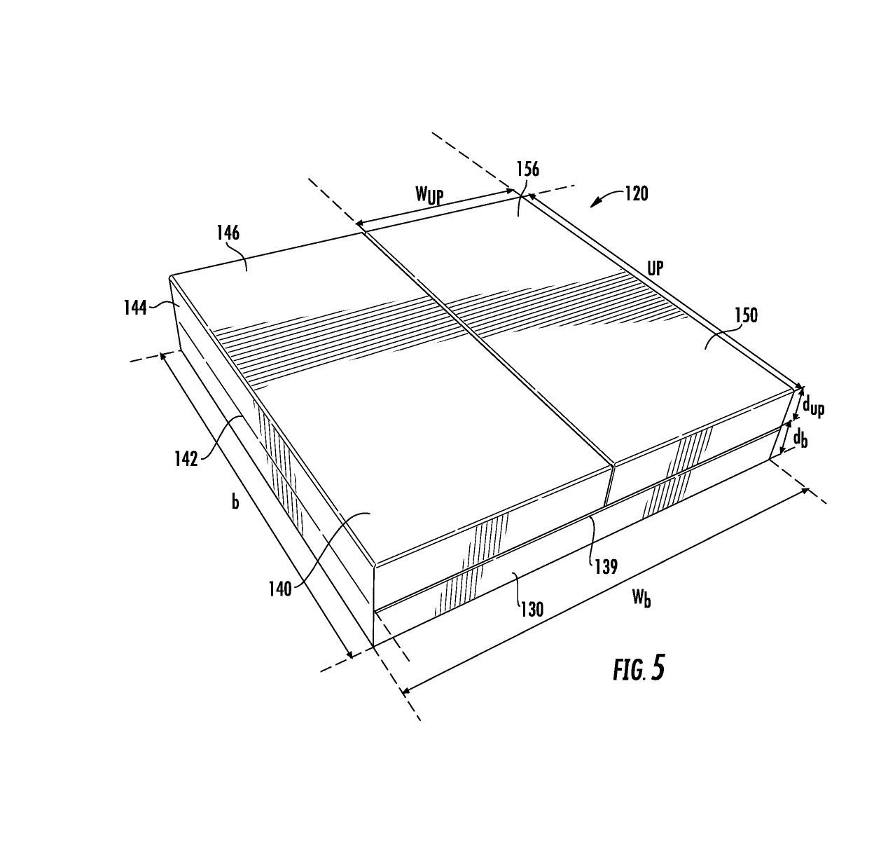

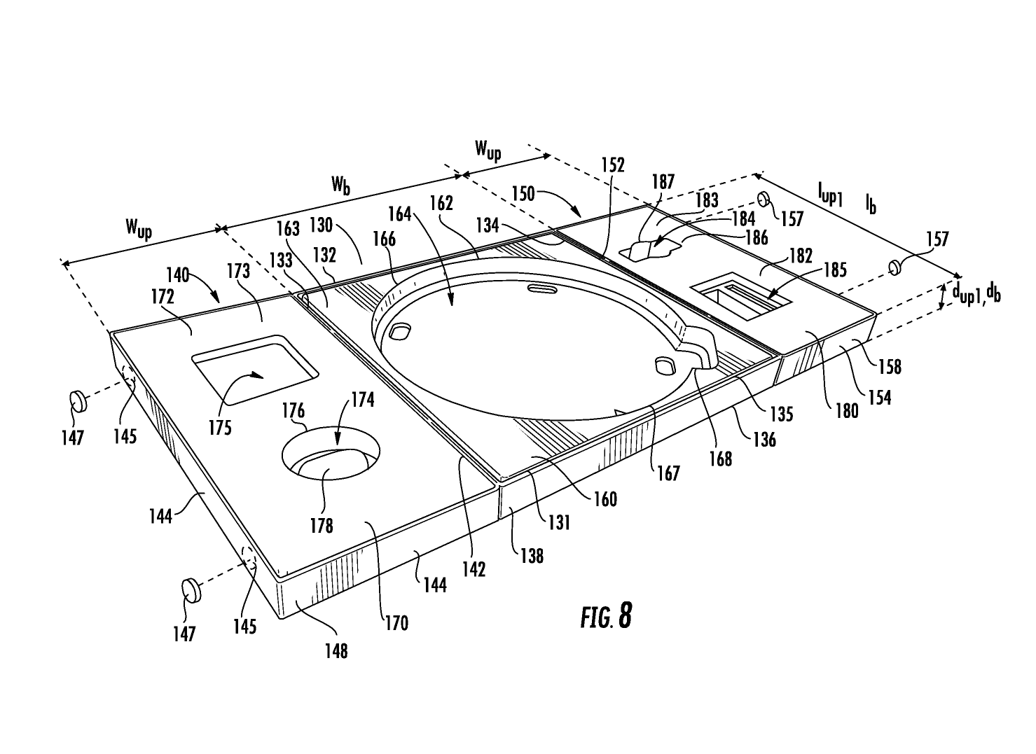

With reference now to FIGS. 1-9, an exemplary embodiment of a health monitoring kit 100 is shown. The health monitoring kit 100 includes an outer lid 110 that is removably positioned on a package unit 120. The package unit 120 includes a base panel 130, a first upper panel 140, and a second upper panel 150. As particularly shown in FIG. 7, a scale 210 is retained in the base panel 130, a display band 220 is retained in the first upper panel 140, and a heart monitor 230 is retained in the second upper panel 150.

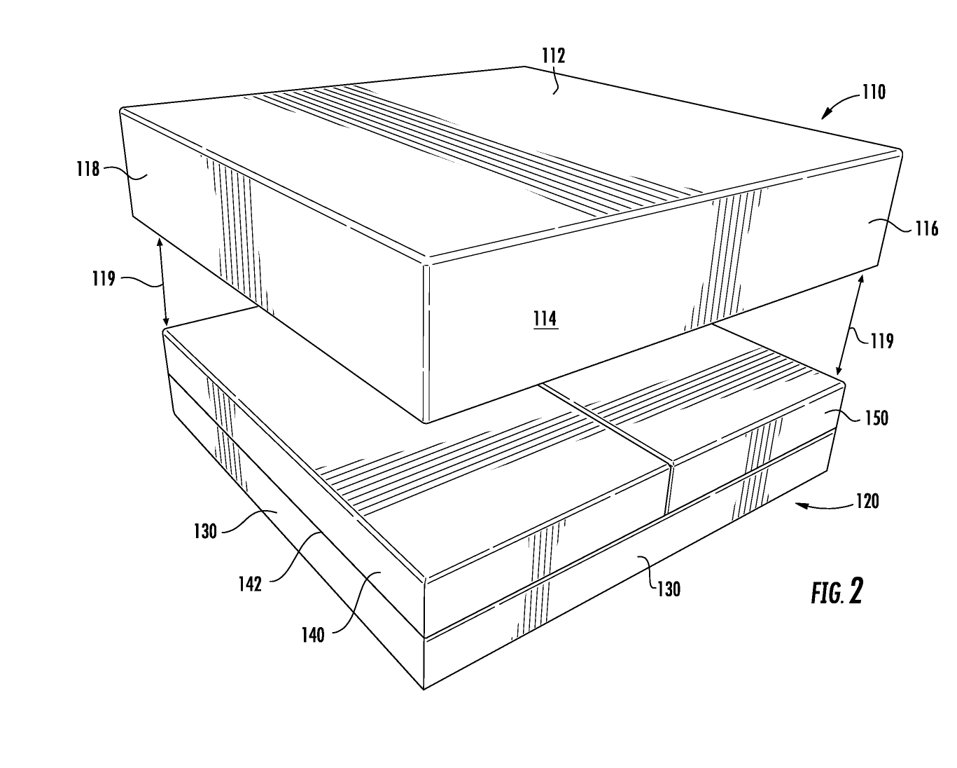

With particular reference to FIGS. 1-4, the outer lid 110 is a box-like structure including a ceiling 112 and four sidewalls 114, including a proximate side wall 116, a left side wall 118, a distal side wall (not shown), and a right side wall (not shown). The outer lid 110 is configured to fit over the package unit 120 such that the ceiling 112 and four sidewalls 114 cover the package unit 120, with only the bottom of the package unit not covered by the outer lid 110. The size and shape outer lid 110 are such that the outer lid 110 may be repeatedly positioned on and then removed from the package unit 120, as noted by arrows 119 in FIG. 2. The outer lid 110 may be comprised of any of various components, such as cardboard, plastic, wood, or metal. In at least one embodiment, the outer lid 110 is comprised of a cardboard material with a polymer outer skin adhered to the cardboard material. The polymer outer skin may include text, graphics or other indicia related to the contents of the health monitoring kit 100.

As shown in FIGS. 2-5, the package unit 120 is provided as a cabinet including a base panel 130, a left upper panel 140, and a right upper panel 150. A left hinge member 142 pivotably connects the left upper panel 140 to the base panel 130, and a right hinge member 152 pivotably connects the right upper panel 150 to the base panel 130. The left hinge member 142 and the right hinge member 152 may be provided by any of various mechanical components pivotably connecting the base panel 130 to one of the upper panels 140, 150. Exemplary hinge members include barrel hinges, pivot hinges, continuous hinges, living hinges, or any of various other types of hinges. As a result of this pivotable connection, the package unit 120 provides a cabinet with the left upper panel 140 provides a left door that pivots relative to the base of the cabinet, and the right upper panel 150 provides a right door that pivots relative to the base of the cabinet.

Similar to the outer lid 110, the package unit 120 may be comprised of any of various components, such as cardboard, plastic, wood, or metal. In at least one embodiment, the package unit 120 is comprised of a cardboard material with a thin outer skin comprised of a polymer material which is adhered to the cardboard material. Although the polymer outer skin is not referenced in the figures, it will be recognized that the polymer outer skin is similar to that provided on other packaging used in commerce. Accordingly, the polymer outer skin may include text, graphics or other indicia related to the contents of the health monitoring kit 100. Moreover, the polymer outer skin may provide the left hinge member 142 by providing a continuous outer layer that extends from the base panel 130 to the left upper panel 140 along the left side of the package unit 120. In like manner, the polymer outer skin may provide the right hinge member 152 by providing a continuous outer layer that extends from the base panel 130 to the right upper panel 150.

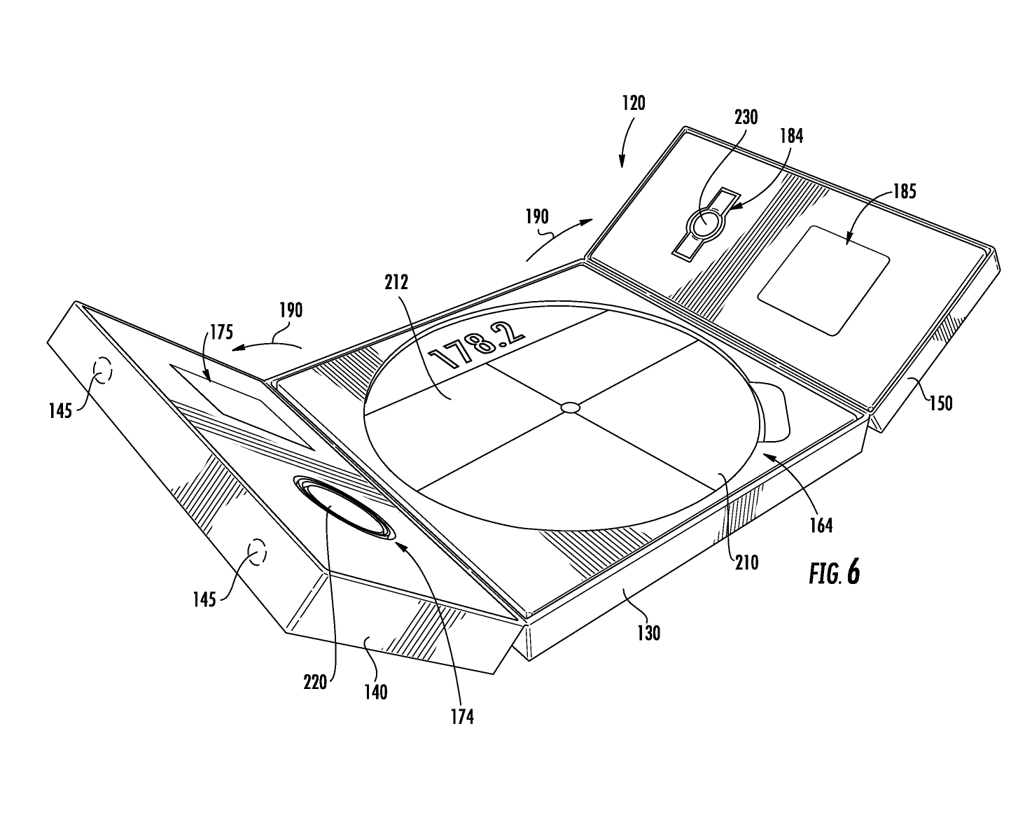

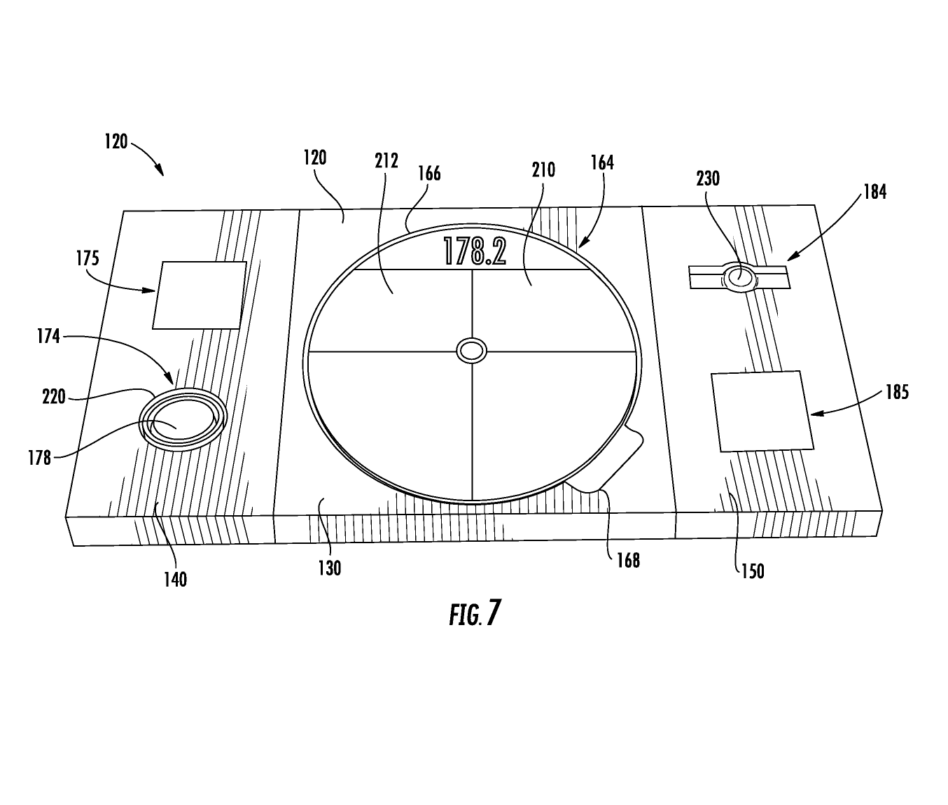

With reference now to FIGS. 5-7, the pivotable configuration of the left upper panel 140 and the right upper panel 150 allows the package unit 120 to be arranged in a closed position, as shown in FIG. 5, or an open position, as shown in FIG. 7. FIG. 6 shows the left upper panel 140 and the right upper panel 150 in transition between the closed position (of FIG. 5) and the open position (of FIG. 7). The arrows 190 of FIG. 6 illustrate the pivoting movement of the left upper panel 140 and the right upper panel 150 moving from the closed position (of FIG. 5) to the open position (of FIG. 7).

When the left upper panel 140 and the right upper panel 150 are in the open position (as shown in FIG. 7), the contents of the package unit 120 are exposed. A number of health tracking devices are retained in the package unit 120 including the scale 210, the display band 220, and the heart monitor 230. The scale 210 is removably retained in the base panel 130. The display band 220 is removably retained in the left upper panel 140. The heart monitor 230 is removably retained in the right upper panel 150. Each of these health tracking devices is explained in further detail below, following further description of the base panel 130, the left upper panel 140, and the right upper panel 150 which retain the health tracking devices.

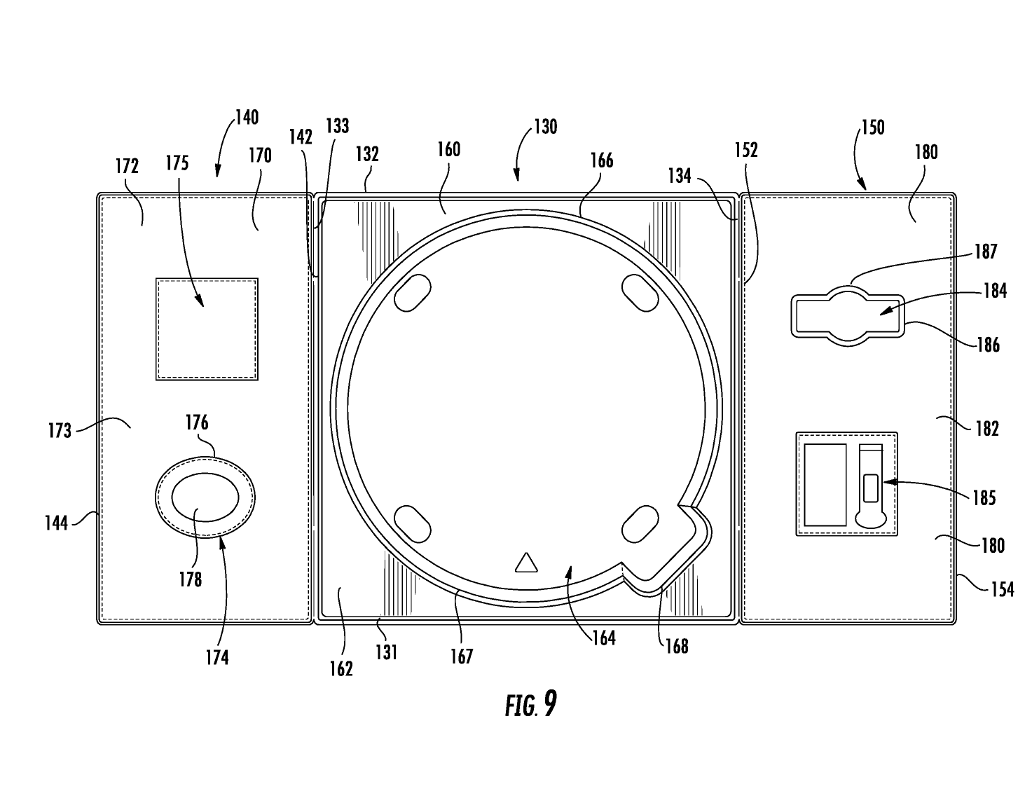

FIGS. 8 and 9 show the package unit 120 with the health tracking devices 210, 220 and 230 removed from the package unit to expose further detail concerning the interior of the base panel 130, left upper panel 140, and right upper panel 150. The base panel 130 has a rectangular cuboid shape, and particularly a square cuboid shape (i.e., a substantially square cross-section). Accordingly, the base panel 130 includes a length l_{b}, a width w_{b}, and a depth d_{b} (also shown in FIG. 5). The length l_{b} extends from a proximate edge 131 to a distal edge 132 of the base panel 130. The width w_{b} extends from a left edge 133 to a right edge 134 of the base panel 130. The depth d_{b} extends from an upper edge 135 to a lower edge 136 of the base panel. The length lb is substantially the same as the width w_{b}, and significantly greater than the depth d_{b}. In at least one embodiment the length l_{b} and the width w_{b} are between 35-45 cm, and the depth d_{b} is between 5 cm and 10 cm. Together, the length l_{b}, width w_{b}, and depth d_{b}, define a volume for the base panel 130.

The base panel 130 includes a base box portion 138 and a base insert 160. The base box portion 138 includes four perimeter walls that define the proximate edge 131, distal edge 132, left edge 133, and right edge 134 of the base panel 130, along with a connecting wall that provides a floor (not shown) extending between the four perimeter walls under the base insert 160. The base box portion 138 may be comprised of any of various components, such as cardboard, plastic, wood, or metal. In at least one embodiment, the base box portion 138 is comprised of a plurality of cardboard sheets with a thin polymer outer skin adhesively connected to the cardboard sheets. As noted previously, the polymer outer skin may provide the left hinge member 142 and right hinge member 152.

The base insert 160 of the base panel 130 is closely received within the base box portion 138. The base insert 160 includes a base face 162 that defines a flat perimeter portion 163 and a scale cavity 164. The flat perimeter portion 163 of the base face 162 is substantially flush with the upper edge of the base box portion 138. The scale cavity 164 of the base face 162 extends into the volume of the base box portion 138 and fills a majority of the volume of the base box portion 138. The scale cavity 164 is configured to receive the scale 210 and is defined by a substantially circular edge 166. The substantially circular edge 166 has a circular portion 167 and a finger recess 168. The circular portion 167 is defined by a radius. The finger recess 168 extends radially outward from the circular portion 167. The finger recess 168 extends across only a relatively small arc of the substantially circular edge 166. In at least one embodiment, the circular portion 167 spans at least three hundred degrees, and the finger recess spans less than sixty degrees. The scale cavity 164 has a depth that is less than the depth of the base box portion 138 but sufficiently deep such that an upper face of the scale 210 is substantially flush with the substantially circular edge 166 when the scale 210 is placed in the scale cavity 164 (as shown in FIG. 7). The finger recess 168 is configured to allow one to four fingers of a human hand to reach into the scale cavity 164 at the finger recess 168 and contact the bottom of the scale 210 when the scale is positioned in the scale cavity 164.

The base insert 160 may be comprised of any of various components, such as cardboard, plastic, wood, or metal. In at least one embodiment, the base insert 160 is comprised of a molded plastic material such as polysterene or other polymer material.

With continued reference to FIGS. 8 and 9, both the left upper panel 140 and the right upper panel 150 are the same size and shape, each having a rectangular cuboid shape. The left and right upper panels 140 and 150 each have a length lᵤₚ, a width wᵤₚ, and a depth dᵤₚ (also shown in FIG. 5). The length lᵤₚ is substantially the same as the length l_{b} of the base panel 130. The width wᵤₚ of the left and right upper panels 140 and 150 is about half the width w_{b} of the base panel 130. The depth dᵤₚ of the left and right upper panels 140 and 150 is substantially the same as the depth d_{b} of the base panel. Together, the length lᵤₚ, width wᵤₚ, and depth dᵤₚ, define a volume for each of the left and right upper panels 140 and 150.

The left upper panel 140 includes a left box portion 148 and a left insert 170. Similar to the base box portion 138, the left box portion 148 includes four perimeter walls 144 and a connecting wall 146 (see FIG. 5) that extends between the four perimeter walls 144. The left hinge member 142 is connected to one of the walls 144 and couples the left box portion 148 to the base box portion 138. The wall opposite the left hinge member 142 includes two recesses 145 with a magnet 147 (shown in exploded isometric form in FIG. 8) positioned in each of the two recess 145. The polymer outer skin covers the four perimeter walls 144 and the connecting wall 146 of the left box portion 148, including the two recesses 145 (shown in dotted lines in FIG. 8) and the magnets 147 therein. Accordingly, the magnets 147 are retained in the recesses 145 by the outer skin.

The left insert 170 of the left upper panel 140 is closely received within the left box portion 148. The left insert 170 includes a left face 172 that defines a flat perimeter portion 173, a band cavity 174, and a pamphlet cavity 175. The flat perimeter portion 173 of the left face 172 is substantially flush with the upper edges of the four walls 144 of the left box portion 148. Both the band cavity 174 and the pamphlet cavity 175 extend into the volume of the left box portion 148, each filling only a minority of the volume.

The band cavity 174 is configured to receive the display band 220 (as shown in FIG. 7) and is defined by a substantially circular outer edge 176 and a central knob 178. The central knob 178 is cylindrical and elliptical or rounded in shape and is designed and dimensioned such that the display band 220 may encompass the central knob 178. When the display band 220 is positioned in the band cavity 174, the band 220 is positioned between the outer edge 176 and the central knob 178.

The pamphlet cavity 175 is a relatively shallow cavity on the left insert 170 having a rectangular cuboid shape. The pamphlet cavity 175 is configured to receive a user's manual, guide, pamphlet, advertising material, promotional material, or other materials or printed matter that may be related to the health monitoring kit 100.

The left upper panel 140, including the left insert 170 and the left box portion 148, may be comprised of any of various components, such as cardboard, plastic, wood, or metal. In at least one embodiment, the left box portion 148 is comprised of cardboard with a polymer skin, and the left insert 170 is comprised of a molded plastic material such as polysterene or other polymer material.

With continued reference to FIGS. 8 and 9, the right upper panel 150 includes a right box portion 158 and a right insert 180. Similar to the left box portion 148, the right box portion 158 includes four perimeter walls 154 and a connecting wall 156 (see FIG. 5) that extends between the four perimeter walls 154. The right hinge member 152 is connected to one of the walls 154 and couples the right box portion 158 to the base box portion 138. The wall opposite the right hinge member 152 includes two recesses (not shown) with a magnet 157 positioned in each of the two recess (similar to the two recesses 145 and magnets 147 of the left box portion 148). The polymer outer skin covers the four perimeter walls 154 and the connecting wall 156 of the right box portion 158, including the two recesses and the associated magnets 157.

The right insert 180 of the right upper panel 150 is closely received within the right box portion 158. The right insert 180 includes a right face 182 that defines a flat perimeter portion 183, a heart monitor cavity 184, and an accessory cavity 185. The flat perimeter portion 183 of the right face 182 is substantially flush with the upper edges of the four walls 154 of the right box portion 158. Both the heart monitor cavity 184 and the accessory cavity 185 extend into the volume of the right box portion 158, each filling only a minority of the volume.

The heart monitor cavity 184 is configured to receive the heart monitor 230 (as shown in FIG. 7). The heart monitor cavity 184 includes two substantially rectangular opposing ends 186 with a rounded central portion 187. When the heart monitor 230 is positioned in the heart monitor cavity 184, a transmitter module of the heart monitor 230 is positioned in the central portion 187, and a strap of the heart monitor 230 is positioned between the two substantially rectangular opposing ends 186.

The accessory cavity 185 is a relatively shallow cavity on the right insert 180 having a generally rectangular cuboid shape with additional indentations for one or more accessories provided with the health monitoring kit 100. For example, the accessory cavity 185 may be configured to receive a battery, secondary strap, or other device that may be used in association with the scale 210, display band 220 or the heart monitor 230. The accessory cavity 185 is further configured to receive a user's manual, guide, pamphlet, advertising material, promotional material, or other materials or printed matter that may be related to the health monitoring kit 100.

The right upper panel 150, including the right insert 180 and the right box portion 158, may be comprised of any of various components, such as cardboard, plastic, wood, or metal. In at least one embodiment, the right box portion 158 is comprised of cardboard with a polymer skin, and the right insert 180 is comprised of a molded plastic material such as polysterene or other polymer material.

Returning now to FIGS. 5-7, operation of the package unit 120 of the health monitoring kit 100 is now described. FIG. 5 shows the package unit 120 with the outer lid 110 removed from the package unit. The package unit 120 is in a closed position with the left upper panel 140 and the right upper panel 150 covering the base panel 130. In this position, the left upper panel 140 extends from the proximal edge to the distal edge of the base panel 130, and from the left edge to a center portion 139 of the base panel. Similarly, the right upper panel extends from the proximal edge to the distal edge of the base panel 130, and from a right edge to the center portion 139 of the base panel 130. As a result, the left upper panel 140 and right upper panel 150 completely cover the base face 162 of the base insert 160. Also, the magnets 147 on the left upper panel 140 and the magnets 157 on the right upper panel 150 are in sufficient proximity that a magnetic coupling exists between the magnets 147, 157. The strength of the magnetic coupling is dependent upon the type of magnets used in the left upper panel 140 and the right upper panel 150. In at least one embodiment, the magnets 147, 157 result in a magnetic coupling that is sufficiently strong to be perceived by a human user when opening the left upper panel 140 and the right upper panel 150, but not sufficiently strong to keep the left upper panel 140 and the right upper panel in the closed position when the package unit is overturned. This magnetic coupling provides a unique tactile experience to the user when opening the package unit 120. It will be recognized that the term “magnet” as used herein refers to a material or device capable of producing a magnetic field, such as permanent magnets, as well as materials attracted to magnetic fields, such as iron or other ferromagnetic material.

FIG. 6 shows the left upper panel 140 and the right upper panel 150 in transition between the closed position (of FIG. 5) and the open position (of FIG. 7). The arrows 190 of FIG. 6 illustrate the pivoting movement of the left upper panel 140 and the right upper panel 150 moving in transition from the closed position (of FIG. 5) toward the open position (of FIG. 7). In this transition position, the health tracking devices for the health monitoring kit 100 are visible, including the scale 210, the display band 220, and the heart monitor 230. The scale 210 is positioned in the scale cavity 164 and substantially fills the scale cavity 164. The display band 220 is positioned in the band cavity 174 and substantially fills the band cavity 174. The heart monitor 230 is positioned in the heart monitor cavity 184, and substantially fills the heart monitor cavity 184.

FIG. 7 shows the left upper panel 140 and the right upper panel 150 in the open position. In this open position, the left upper panel 140 and the right upper panel 150 are laid open and are substantially flush with the base panel 130, thus providing a surface wherein the contents of the kit are easily available for easy viewing and removal from the package unit 120. For example, the kit 100 may be displayed in a store with the package unit 120 in the open position, thus allowing potential purchasers to see the contents of the kit 100. Additionally, the depth d_{b} of the base panel 130 and the depth dᵤₚ of the upper panels is sufficiently wide such that the package unit 120 may be stood on end for display. After the kit 100 has been purchased, the user may easily place the package unit 120 in the open position, and reach into the kit and remove any of the scale 210, the display band 220, or the heart monitor 230.

The scale 210 is configured to weigh a human body when the human stands on the scale 210. The scale 210 includes a relatively flat face 212 configured to receive the feet of a human user. In the embodiment disclosed herein, the scale has a generally circular cylindrical shape such that it fits closely within the scale cavity 164 of the base panel. When the scale 210 is removed from the base panel 130 and provided on a floor or other flat surface, the user may stand on the scale and receive a weight measurement. The scale 210 may be configured with any of various functions, such as weight measurement and/or body fat measurement. In at least one embodiment, the scale 210 is configured for wireless communications with the display band 220 or other health tracking device such that any measurements taken with the scale are automatically transmitted to the associated health tracking device. After use, the user may return the scale 210 to the scale cavity 164 in the package unit 120 for safekeeping.

The display band 220 is configured to be worn on the body of a human, such as on the wrist of a human. The display band 220 is further configured to track activity of the user such as steps taken, distance travelled, time of travel, or hours slept. The display band includes a display that is configured to present any of various health-related metrics to the user. The display may also be configured to provide other information to the user, such as time of day, thus providing a watch for the user. In at least one embodiment, the display band 220 is configured for wireless communication with the scale 210, the heart monitor 230, and any of various other health tracking devices, such as a smartphone. This wireless communication allows the metrics received with each of the various health tracking devices to be assembled presented in a single location (e.g., on the display band 220 or the smartphone). In at least one embodiment, the display band 220 is a smartwatch capable of communicating with a smartphone or other device. During times when the user does not wear the display band 220, the band cavity 174 provides a convenient storage location for the display band 220.

The heart monitor 230 is configured to be worn on a human chest and track the heart rate of the user. The heart monitor 230 includes an elastic band with electrodes as well as a transmitter module coupled to the elastic band. The heart monitor 230 is configured to detect the heart rate of the user and transmit the heart rate to a nearby health tracking device, such as the display band 220, or a smartphone. During times when the heart monitor 230 is not in use, the heart monitor cavity 184 provides a convenient storage location for the heart monitor 230.

The foregoing detailed description of one or more exemplary embodiments of the health monitoring kit has been presented herein by way of example only and not limitation. It will be recognized that there are advantages to certain individual features and functions described herein that may be obtained without incorporating other features and functions described herein. Moreover, it will be recognized that various alternatives, modifications, variations, or improvements of the above-disclosed exemplary embodiments and other features and functions, or alternatives thereof, may be desirably combined into many other different embodiments, systems or applications. Presently unforeseen or unanticipated alternatives, modifications, variations, or improvements therein may be subsequently made by those skilled in the art which are also intended to be encompassed by the appended claims. Therefore, the spirit and scope of any appended claims should not be limited to the description of the exemplary embodiments contained herein.

What is claimed is:

1. A health monitoring kit comprising:
a package unit including a base panel, a first upper panel, and a second upper panel, the base panel with a base face, a first upper panel connected to the base panel with a first hinge member, and the second upper panel connected to the base panel with a second hinge member;
a scale retained in the base panel and covering a portion of the base face;
a display band retained in the first upper panel; and
a heart monitor retained in the second upper panel.

2. The health monitoring kit of claim 1 wherein the scale covers most of the base face, the first upper panel is a left upper panel, the first hinge member is a left hinge member, the second upper panel is a right upper panel, and the second hinge member is a right hinge member.

3. The health monitoring kit of claim 2 wherein the left upper panel and the right upper panel are moveable between an open position and a closed position, wherein the right upper panel covers a portion of the base face extending from a proximal edge to a distal edge of the base panel and from a left edge to a center portion of the base panel when the right upper panel is in the closed position, and wherein the right upper panel covers a portion of the base face extending from the proximal edge to the distal edge of the base panel and from a right edge to the center portion of the base panel when the left upper panel is in the closed position.

4. The health monitoring kit of claim 3 wherein the left upper panel and the right upper panel are substantially flush with the base panel when the left upper panel and the right upper panel are in the open position.

5. The health monitoring kit of claim 3 wherein a right side of the left upper panel includes a first magnet, and a left side of the right upper panel includes a second magnet such that a magnetic coupling exists between the first magnet and the second magnet when the left upper panel and the right upper panel are both in the closed position.

6. The health monitoring kit of claim 3 wherein the base panel has a substantially square cuboid shape, wherein the left upper panel and the right upper panel have a substantially rectangular cuboid shape, and wherein the left upper panel is substantially in size to the right upper panel.

7. The health monitoring kit of claim 6 wherein the base panel is defined by a first depth and the first upper panel and the second upper panel are defined by a second depth, the first depth substantially equivalent to the second depth.

8. The health monitoring kit of claim 1 wherein the base face defines a scale cavity with the scale received within the scale cavity, the first upper panel includes a band cavity with the display band received within the band cavity, and the second upper panel includes a heart monitor cavity with the heart monitor received within the heart cavity.

9. The health monitoring kit of claim 8 wherein the scale cavity is defined by substantially circular edge on the base face, the scale including a substantially flat scale face that is flush with the base face at the substantially circular edge, wherein the substantially circular edge of the scale cavity includes a circular portion and a finger recess, the circular portion spanning more than three hundred degrees and the finger recess extending radially outward from the circular portion.

10. The health monitoring kit of claim 8 wherein the band cavity is defined by a substantially circular edge.

11. The health monitoring kit of claim 8 wherein the base panel includes a base box portion and a base insert with the scale cavity provided in the base insert, wherein the first upper panel includes a first box portion and a first insert positioned in the first box portion with the band cavity provided in the first insert, and wherein the second upper panel includes a second box portion and a second insert positioned in the second box portion with the heart monitor cavity provided in the second insert.

12. The health monitoring kit of claim 8 wherein the first upper panel also includes a pamphlet cavity, wherein the heart monitor cavity includes two substantially rectangular opposing ends with a rounded central portion, wherein a transmitter module of the heart monitor is positioned in the central portion, and wherein a strap of the heart monitor is positioned between the two substantially rectangular opposing ends.

13. The health monitoring kit of claim 1 wherein the display band is configured to be worn on a human wrist.

14. The health monitoring kit of claim 1 wherein the base panel has a substantially square cuboid shape and the scale has a circular cylindrical shape defining a diameter of the scale, wherein the diameter of the scale is substantially the same as a length of a side of the base panel.

15. The health monitoring kit of claim 1 wherein the first hinge member is a living hinge or an adhesive strip.

16. The health monitoring kit of claim 1 wherein the scale and the heart monitor are configured for wireless communication with the display band or a personal electronic device.

17. A health monitoring kit comprising:
a cabinet including a base, a first door, and a second door, the base defining a base volume, the first door defining a first door volume, and the second door defining a second door volume;
a first door insert provided in the first door, the first door insert including a first cavity that fills a minority of the first door volume;
a second door insert provided in the second door, the second door insert including a second cavity that fills a minority of the second door volume;
a base insert provided in the base, the base insert including a third cavity that fills a majority of the base volume;
a first health tracking device positioned in and filling a majority of the first cavity;
a second health tracking device positioned in and filling a majority of the second cavity; and
a third health tracking device positioned in and filling a majority of the third cavity.

18. The health monitoring kit of claim 17 wherein the first health tracking device is a heart rate monitor, the second health tracking device is a wristwatch, and the third health tracking device is a scale.

19. A health monitoring kit comprising:
a package unit including a base panel, a first upper panel, and a second upper panel, the first upper panel connected to the base panel with a first hinge member, and the second upper panel connected to the base panel with a second hinge member;
a first health tracking device retained in the base;
a second health tracking device retained in the first upper panel; and
a third health tracking device retained in the second upper panel.

20. The health monitoring kit of claim 19 wherein the first health tracking device is a heart rate monitor, the second health tracking device is a wristwatch, and the third health tracking device is a scale.
